(19) United States
(12) Patent Application Publication
(10) Pub. No.: US 2012/0202210 A1
(43) Pub. Date: Aug. 9, 2012

(54) Biohazard detection system with exhaust stream recycling

(75) Inventors: Eric Gregory Burroughs, Reisterstown, MD (US); Kenneth Scott Damer, Mount Wolf, PA (US); Edmond Grant Radcliff, Laurel, MD (US)

(73) Assignee: Northrop Grumman Systems Corporation, Los Angeles, CA (US)

(21) Appl. No.: 11/892,068

(22) Filed: Aug. 20, 2007

(51) Int. Cl. C12Q 1/68; G01N 27/26; B01D 57/00

(52) U.S. Cl. 435/6.12; 422/527; 205/789

(57) ABSTRACT

A system capable of detecting low-level releases of bio-agents and other harmful substances contained in air concentrates and recycles exhaust air from a collector in order to entrain and retain more particles from an aerosol release event. The system ensures that particles that were not initially entrained or subsequently retained will have successive opportunities to be entrained within the collection solution. The system also optionally utilizes concentrators in series and a collector to ensure that the particle content of air entering the collector is maximized.

### BACKGROUND OF THE INVENTION

1. Field of the Invention

The present invention relates generally to biohazard detection systems, aerosol collection technology and, more particularly, to a system and method for enhanced detection of aerosolized biohazards.

2. Discussion of Related Art

Biohazard detection has become very important to society because of the increased threat of terrorist attacks. Fears that terrorists have a multitude of weapons at their disposal, including chemical and biological weapons, have increased in view of recent events. For example, following the Sep. 11, 2001 attacks by Al-Qaida on the World Trade Center in New York City, Anthrax spores were introduced into parcels circulated by the U.S. Postal Service, causing injury and death.

Anthrax spores typically appear as a powdery substance to the naked eye, and can become suspended in air (i.e., aerosolized) when agitated. When suspended in air, the spores are not visible to the naked eye and cannot otherwise be sensed by humans. Therefore, the undetectable substance can be easily inhaled. Once inhaled, the spores attach to the victim's lungs and wreak havoc within the victim's body causing injury and death. In many cases, the victim will not have knowledge of the inhalation until their physical symptoms become acute.

Anthrax can be weaponized in any number of ways, including introducing the spores into the air ventilation systems of buildings, where the spores are allowed to circulate and increase the chances innocent people will inhale the deadly substance. Of course, Anthrax is but one example of a substance that terrorists can weaponize. Other air-borne chemicals and particles could be used as well.

Biohazard detection systems such as the BDS developed by Northrop Grumman Corporation for the United States Postal Service (described in U.S. Patent Application Publication Nos. 2004/0063198 and 2004/0063197, the contents of which are incorporated herein by reference), have been used to detect the presence of air-borne particles. As shown schematically in FIG. 3, the BDS system 300 includes a particle collection device 302 that collects particles from an input air stream 304(a) for testing by a particle analyzer device 310. An example of a particle collection device is disclosed in U.S. Pat. No. 5,011,517. The '517 patent describes a device, referred to herein as a “collector”, that collects chemical and/or other particulates contained within an air sample. The collector pulls an air sample into a particle collection mechanism in the form of a chamber and forces the chemical vapors and/or other particulates contained within the air sample to come into contact with fluid within the chamber. The fluid captures (i.e., entrains) portions of the particulates within the air sample. After the collector operates for a period of time, the solution containing the particulate can be periodically tested by the particle analyzer device 310 for the presence of a given type of particulate. The SpinCon® Advanced Air Sampler, manufactured by Sceptor Industries, Inc., is an example of such a collector. It should be clear to one of ordinary skill in the art that the subject invention will work with any of a number of aerosol collection devices.

As indicated, such prior art collectors can collect aerosols and aerosolized bioagents such as Anthrax spores contained in an air sample. While this is the case, the sensitivity of the detection equipment is often such that the number of particles collected is not sufficient to obtain an accurate identification, resulting in a failure to detect the harmful agent.

The concentration of particles in a test sample collected by aerosol collection technologies such as the type described above is plagued by two major inefficiencies. Typically, less than half of the particles in the airflow are transferred into the water collection solution (i.e., entrained), while the rest of the particles are carried away by the exhaust stream 304(b). Additionally, continuous airflow through the water solution can cause particles previously entrained in the solution to re-aerosolize (i.e., become airborne once again). Long collection durations can dramatically reduce the retention of particles within the sample as a large volume of air flows through the collection solution. Accordingly, the overall prior art systems have low collection and retention efficiencies because they lose particles that are not initially entrained or lost due to re-aerosolization. If the number of particles collected is not sufficient for the detection equipment to identify the type of particle, it is possible that a biohazard may go unnoticed until a person exposed to the biohazard develops physical symptoms.

What is desired, therefore, is an aerosol collection and analysis system that collects and retains the maximum amount of aerosols. This will result in a more sensitive biohazard detection system capable of detecting even a minute amount of chemicals, aerosols, and/or bio-agents present in air.

### SUMMARY OF THE INVENTION

In accordance with a first aspect of the present invention, a biohazard detection system includes a collector with a particle collection mechanism configured to remove particles from a first stream of fluid to be tested, and a concentrator with a particle concentration mechanism configured to concentrate particles contained within the fluid expelled from the collector for combination with the first stream of fluid. The collector includes a first fluid inlet in communication with the first stream of fluid having a first concentration of particles, and a first fluid outlet configured to expel at least some of the fluid from the particle collection mechanism as a second stream of fluid having a second concentration of particles lower than the first concentration of particles. The concentrator includes a second fluid inlet in communication with the first fluid outlet to receive the second stream of fluid, and a second fluid outlet communicating a third stream of fluid from the particle concentration mechanism with the first fluid inlet such that the third stream of fluid is combined with the first stream of fluid entering the collector to improve system performance by boosting the concentration of particles entering the collector. A testing instrument is configured to receive particles from the collector and to test the particles for the presence of a biohazard, aerosols, or chemicals.

In accordance with a second aspect of the present invention, a biohazard detection system includes first and second concentrators with particle concentration mechanisms and a collector with a particle collection mechanism. The first concentrator includes a first fluid inlet in communication with a first stream of fluid having a first concentration of particles to be tested, a first fluid outlet configured to expel a second stream of fluid with a second concentration of particles from the particle concentration mechanism, and a second fluid outlet configured to expel a third stream of fluid with a third concentration of particles lower than the second concentration of particles from the particle concentration mechanism. The second concentrator includes a second fluid inlet in communication with the second fluid outlet to receive the third stream of fluid, and a third fluid outlet configured to expel a fourth stream of fluid from the second particle concentration mechanism. The collector receives the second stream of fluid from the first concentrator, and expels at least some of the fluid. The fourth stream of fluid from the second concentrator is combined with the first stream of fluid entering the first concentrator so that the concentration of particles in the fluid entering the collector is enhanced. A testing instrument is configured to receive particles from the collector and to test the particles for the presence of a biohazard.

In accordance with a third aspect of the present invention, a biohazard detection method includes the steps of receiving in a collector a first stream of fluid having a first concentration of particles; collecting particles from the first stream of fluid using the collector; expelling at least some of the fluid from the collector as a second stream of fluid having a second concentration of particles lower than the first concentration of particles; receiving the second stream of fluid in a concentrator; using the concentrator to produce from the second stream of fluid a third stream of fluid with a third concentration of particles greater than the second concentration of particles and a fourth stream of fluid with a fourth concentration of particles lower than the third concentration of particles; and combining the third stream of fluid with the first stream of fluid to enhance the concentration of particles in the fluid entering the collector. The method also includes the step of testing particles collected during the collecting step for the presence of a biohazard.

In accordance with a fourth aspect of the present invention, a biohazard detection method includes the steps of receiving in a first concentrator a first stream of fluid having a first concentration of particles; using the first concentrator to produce from the first stream of fluid a second stream of fluid with a second concentration of particles greater than the first concentration of particles and a third stream of fluid with a third concentration of particles lower than the second concentration of particles; receiving in a second concentrator the third stream of fluid; using the second concentrator to produce from the third stream of fluid a fourth stream of fluid with a fourth concentration of particles greater than the third concentration of particles and a fifth stream of fluid with a fifth concentration of particles lower than the fourth concentration of particles; combining the fourth stream of fluid with the first stream of fluid received by the first concentrator; receiving in a collector the second stream of fluid produced by the first concentrator; and collecting particles from the second stream of fluid using the collector such that the concentration of particles in the second stream is enhanced. The method also includes the step of testing particles collected during the collecting step for the presence of a biohazard.

The above and other features and advantages of the present invention, as well as the structure and operation of preferred embodiments of the present invention, are described in detail below with reference to the accompanying drawings.

### BRIEF DESCRIPTION OF THE DRAWINGS

The accompanying drawings, which are incorporated herein and form part of the specification, help illustrate various embodiments of the present invention and, together with the description, further serve to explain the principles of the invention and to enable a person skilled in the pertinent art to make and use the invention. In the drawings, like reference numbers indicate identical or functionally similar elements.

FIG. 1 is a schematic diagram of a biohazard detection system with exhaust stream recycling according to a first embodiment of the present invention.

FIG. 2 is a schematic diagram of a biohazard detection system with exhaust stream recycling according to a second embodiment of the present invention.

FIG. 3 is a schematic diagram of a prior art detection system.

FIG. 4 is a schematic diagram of another prior art detection system.

FIG. 5 is a plot of fraction transported as a function of concentrator number and efficiency.

FIG. 6 is a plot of boost in system performance as a function of concentrator number and efficiency.

FIG. 7 is a plot of concentrator efficiency as a function of fraction and number of additional concentrators.

### DETAILED DESCRIPTION OF PREFERRED EMBODIMENTS

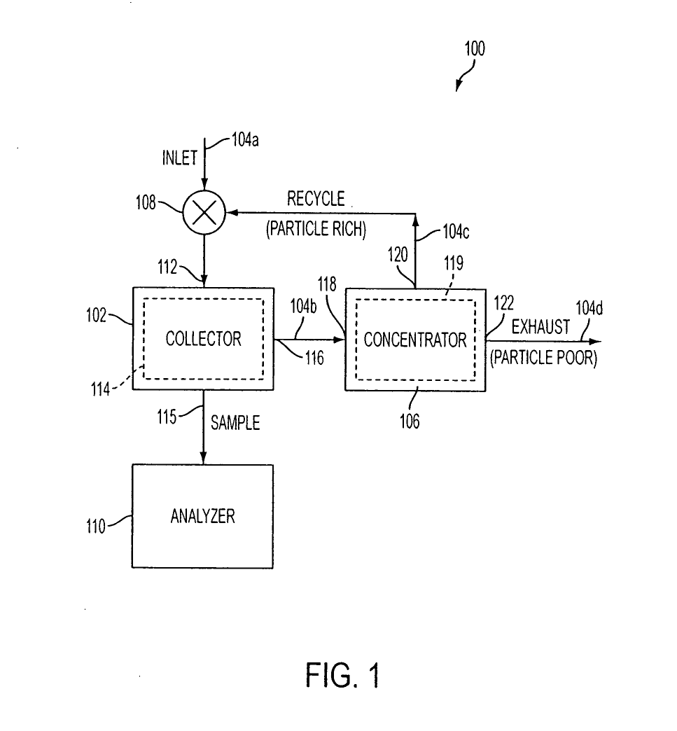

FIG. 1 shows a system 100 for detecting aerosols, chemicals, bio-agents and other harmful substances (hereinafter collectively referred to as “biohazards”) according to a first embodiment of the present invention. The biohazard detection system 100 includes a collector 102 configured to collect particles from an input air stream 104(a) and exhaust a waste air stream 104(b), a concentrator 106 configured to recover particles from the waste air stream 104(b) and reintroduce the particles as a concentrated recycled air stream 104(c) into the input air stream via a mixer 108. The system 100 also includes a device 110 configured to analyze the particles collected by the collector 102 for the presence of biohazards.

The collector 102 includes an air inlet 112, a particle collection mechanism 114, a sample output 115, and an air outlet 116. Air inlet 112 of the collector 102 is in communication with input air stream 104(a) and collector mechanism 114. The input air stream 104(a) can be directed to the system 100 directly via the inlet 112 or through some type of interface, such as a hood, nozzle, duct or other fluid conduit. The collector may optionally include a blower (not shown) to create a vacuum and circulate air throughout the system 100. In a preferred embodiment, the collector mechanism 114 includes a chamber containing fluid, such as water, and the air inlet 112 communicates with openings in the chamber wall oriented to create a vortex when air is drawn into the collector. The SpinCon® Advanced Air Sampler manufactured by Sceptor Industries is an example of such a collector, although other types of collectors can be used.

The concentrator 106 is designed to concentrate the waste air stream 104(b) exhausted from collector 102 into a smaller volume. This reduction in air volume creates a particle-rich stream of air 104(c) that can be recycled into the system, and a particle-poor stream of air 104(d) that can be expelled from the system. The concentrator 106 includes an air inlet 118, and a particle concentration mechanism 119 that produces the concentrated and particle-poor stream of air, a concentrated air outlet 120 and an exhaust air outlet 122. Examples of commercial concentrators that can be used in the system include, but are not limited to, concentrating virtual impactor (CVI) type concentrators, such as the MSP Model 4220 Aerosol Concentrator manufactured by MSP Corporation of Shoreview, Minn., and the Micro VIC particle concentrator manufactured by icx Mesosystems of Albuquerque, N. Mex.

The mixer 108 is positioned upstream of the collector 102 and can be any type of valve, coupling, or manifold capable of combining the recycle stream 104(c) and the input stream 104(a) into a single input stream that feeds into the collector. For example, the mixer 108 can include, but is not limited to, “T” and “Y”-shaped couplings and venturi-type manifolds that prevent backflow.

In operation, when system 100 is powered, a vacuum is created within the collector chamber 114, causing the input air stream 104(a) to be drawn into the collector chamber 114 through the collector air inlet 112. In a vortex-type collector, the air is pulled into the chamber 114 at an angle, relatively tangent to the circumference of the chamber 114, through a plurality of openings, thereby creating a vortex within the chamber 114. That is, when the air sample is pulled within the chamber 114, the air circulates around the inner circumference of the chamber 114, creating a circulating vortex of air. As the air circulates around the inside of the chamber 114, the force of the air causes the fluid in the chamber to circulate around the inside of the chamber 114 as well. As this process continues, the air makes contact with the fluid, and some of the particles contained within the air become trapped by the fluid.

As the collector 102 operates, the air sample being drawn into the chamber 114 interacts with the collection mechanism. As new air comes into the system 100, air already contained in the chamber 114 exhausts through the collector 102 air outlet 116. The input air stream 104(a) will have a greater concentration of particulate than the exhaust air stream 104(b) leaving the collector 102 through the collector outlet 116. In any event, as the first cycle of the collector 102 is imperfect, the exhaust air stream 104(b) exiting through the collector air outlet 116 will contain particles not successfully entrained within the collector 102.

The exhaust air stream 104(b) leaving the collector 102 through the collector outlet 116 enters the concentrator 106 via the concentrator air inlet 118. After the concentrator 106 receives the exhausted air sample from the collector 102, the concentrator 106 reduces that air sample into a smaller volume, thereby creating a concentrated particle-rich stream of air 104(c). The particle-rich stream of air 104(c) is then channeled through the concentrator outlet 120 and recycled back into the collector 102 via the mixer or manifold 108 which combines the concentrated stream of air 104(c) with the inflow air stream 104(a). That is, the air outlet 120 of the concentrator 106 is in communication with the air inlet 112 of the collector 102, allowing particle rich air to re-circulate back into the collector 102. As this process continues, particles that were not initially entrained or subsequently retained will have successive opportunities to be entrained within the collection mechanism of the collector 102.

The concentrator 110 also has an exhaust 122 that rids the system of particle poor-air—i.e. air that contains very small amounts of particles through a particle—poor air stream 104(d). Therefore, as the system 100 operates, a continuous flow of new air sample enters the collector 102 as a continuous flow of particle-poor volume of air exhausts the system 100 through the concentrator exhaust 122.

Recycling a particle rich portion of the effluent stream substantially boosts the number of particles entrained and retained within the collection solution of the collector 102. In this manner, the overall system 100 collection efficiency and the entrainment and retention efficiencies of the system 100 are greatly enhanced over the prior art and the system 100 will ultimately be more sensitive to the detection of a chemical or bio-agent release. Doing so can dramatically enhance, upwards of 10 fold, the ability of the system 100 to entrain and retain an aerosol release event.

To quantify the recycling concept's impact on the overall system 100 collection efficiency and the entrainment and subsequent retention efficiencies, the system can be described using material (mass) balances. Materials must be conserved within a system and, as such, equations can be formulated that account for the inlets, outlets, generation (re-aerosolization of the particles entrained in the collector), and consumption (particles entrained in the collector). These equations include both temporally dependant and steady-state material balances over the collection system as a whole and each processing unit (collector, concentrator, and mixing points).

Once the systems are mathematically described, efficiencies can be computed to quantify how well the system 100 is entraining and retaining the aerosol event. The amount of particles entrained by the system 100 relative to the amount of particles in the aerosol event referred to herein as the entrainment efficiency. The relative amount of particles retained by the collection mechanism, compared to the amount initially entrained, is referred to herein as the retention efficiency. Finally, the relative amount of particles in the sample, when tested at time t, compared to the total amount of particles in the aerosol event is referred to herein as the overall system 100 collection efficiency.

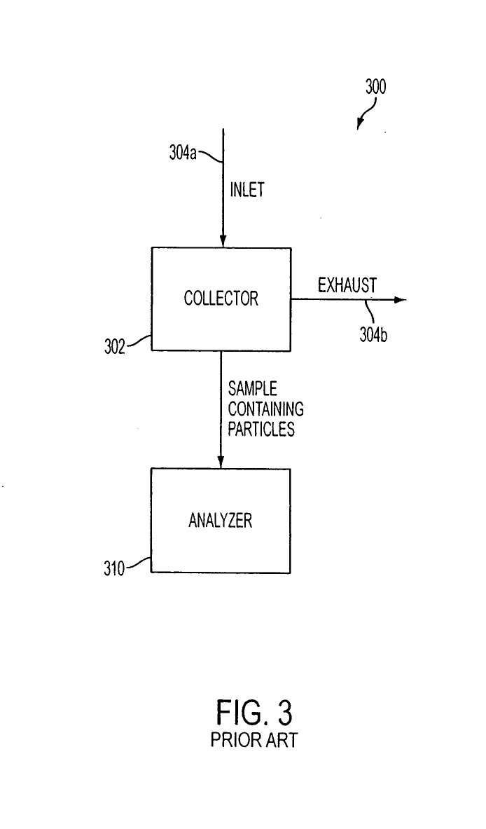

Applying this mathematical approach to the prior art detection system 300 of FIG. 3, and assuming no aerosol loss by deposition in the lines, the entrainment and subsequent retention of an aerosol release event is described by Equations 1 and 2, below:

$$\frac{d m}{d t} = - k m ⇒ {m}_{{t}_{f}} = {m}_{{t}_{0}} {e}^{- kt} \qquad Eqns . 1 \& 2$$

In concept, these equations simply state that the amount of mass entrained and retained by the system, m_{tf} is the amount of mass from the aerosol event that is initially entrained in the sample solution, mₜₒ, reduced by the re-aerosolization of entrained particles. The re-aerosolization is exponentially decaying at a certain rate, given by k. Note that in the absence of loss by re-aerosolization, the exponent term equals unity, such that the overall system collection efficiency is equal to the entrainment efficiency.

Applying the same approach to a system 100 according to the present invention as shown in FIG. 1, the entrainment and retention of an aerosol release event is described by Equation 3, below:

$$\frac{d m}{d t} = λ {Q}_{i} ( m , t ) - [ 1 - β ] k m ( t ) \qquad Eqn . 3$$

The first term on the right represents the mass of the aerosol release event that is entrained in the collection mechanism, whereas the second term represents the mass lost due to the re-aerosolization of the particles entrained in the collection mechanism. λ and β are a combination of the collector/separation efficiencies of the collector/concentrator. Comparing Eqns. 3 and 1, Eqn. 3 has a modifier to the loss term that represents the mass being recycled within the system 100 and thereby retained. In essence the β term dampens the loss term (if there were no recycling, β=0) resulting in more mass being retained within the system 100. Similarly, the mass of the aerosol release event that is entrained in the collection mechanism (first term on the right in Eqn. 3) is enhanced by the recycling (λ enhances). As a simple verification of the analysis, if there were no recycling, λ and β would equal zero such that Eqn. 1 would be retained.

Eqn. 3 is a non-linear equation and, as such, can only be solved numerically when representing a real system. If simple assumptions are made, however, the equation can be directly solved so as to provide an approximation of the impact recycling has on entrainment and retention. In addition, if we assume “typical” operating conditions, we can quantify the various efficiencies. For discussion purposes, we assume a collector efficiency of 40%, a retention efficiency of 50% per hour, and downstream concentrator efficiency of 90%. A basis of 100 grams of spores can be assumed as the equations and quantities scale.

Several measures of performance enhancement can be quantified. First, the entrainment efficiency can be ascertained if the retention is assumed to be perfect (i.e. no loss due to re-aerosolization), or when the collection period is short enough where the losses from re-aerosolization are negligible. For the standard system (no recycling), this would result in 40% of the aerosol release event being entrained in the collection mechanism. By using a recycling system, about 87% of the event is entrained, a two fold boost.

Typically the entrainment of aerosol release events occurs much faster than loss by re-aerosolization. If this is assumed within the mathematical construct, the retention of the entrained particles can be predicted. Applying this assumption to Eqn. 2, the standard system 100 would retain only 50% (20 grams) of the 40 g of entrained aerosols after an hour (25% after 2 hours, and 12.5% after 3 hours), resulting in an overall system 100 collection efficiency of only 20% after 1 hour—20 grams out of 100 grams entering the system—(10% after 2 hours, 5% after 3 hours). To provide a suitable comparison of the recycling system's 100 ability to retain reaerosolized particles, Eqn. 3 can be initialized with 40% entrainment of the aerosol release event (i.e. only recycle the particles that were re-aerosolized from the collection mechanism). With this assumption, adding the recycling line will boost the retention of the aerosol event to 86% (34.4 grams) after an hour (74% after 2 hours, 64% after 3 hours), resulting in an overall system 100 collection efficiency of 34% after an hour (30% after 2 hours, 25% after 3 hours). This results in a 5 fold boost after 3 hours.

Lastly, we can quantify the impact of recycling on the full system by assuming entrainment of the aerosol release event with recycling occurs much faster than the re-aerosolization of the entrained particles. With this assumption, the system initially entrains about 87% of the aerosol release event. The entrained particles are then re-aerosolized and subsequently recycled through the system. After an hour, about 75 grams remain in the system 100, for an overall system collection efficiency that captures 75% of the inlet aerosol event. This is nearly a four fold increase over the standard system which captures only 20% of the event after an hour. Over longer sampling periods, recycling dramatically boosts retention of the aerosol event. For example, after three hours, the recycling system 100 has an overall collection efficiency of 55% versus 5% for the standard system, a ten fold boost.

This gain in performance, particularly for longer collection intervals, has many potential benefits. In addition to the obvious system performance advantages (most notably increased system sensitivity), the increase in retention performance also allows the system to collect for longer periods of time while still retaining an equivalent portion of the entrained aerosol. This potentially reduces operational costs, as the sample can be tested less frequently while still maintaining an equivalent sensitivity.

Of course, these are theoretical limits. A real system would have additional losses due to deposition in the lines that the aerosols flow through. In addition, specific designs of the collector and concentrator would result in different efficiencies (better or worse). Regardless, the boost in overall system collection efficiency and the entrainment and retention efficiencies with a recycling line are dramatic, especially when testing the collection solution over longer collection intervals.

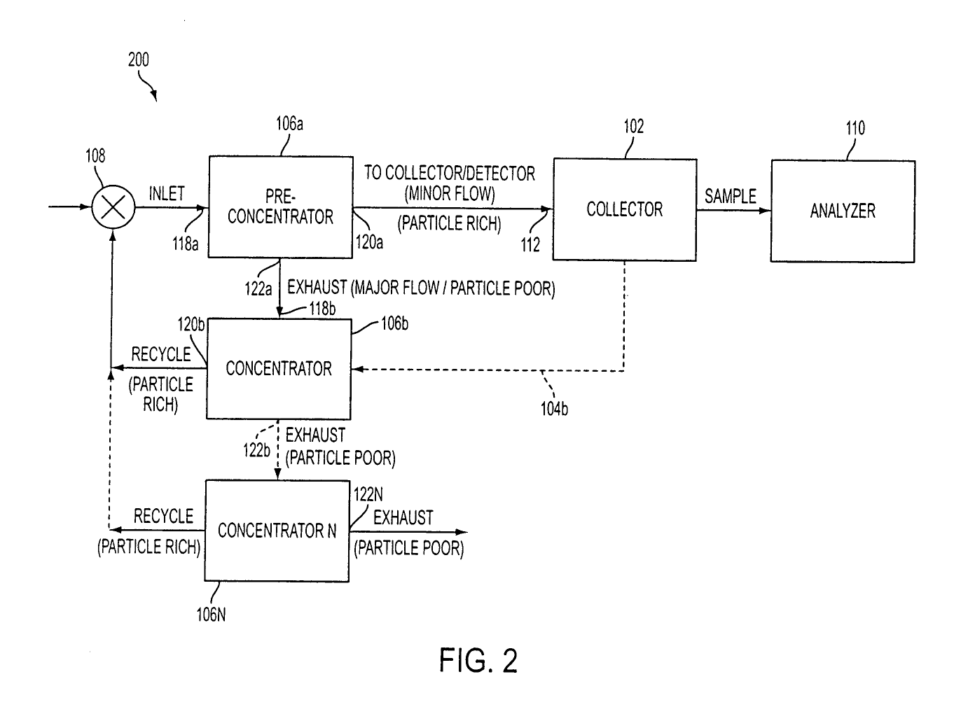

Another embodiment of a biohazard detection system according to the present invention is shown in FIG. 2 at 200. This embodiment is similar to the embodiment described above and shown in FIG. 1, but utilizes a series of concentrators 106(a) and (b) in combination with the collector 102 to ensure that the particle content of air entering the collector 102 is maximized. More specifically, the system 200 contains a second concentrator 106(a) which is disposed upstream of the collector 102. The second concentrator (or pre-concentrator) 106(a) has an inlet 118(a) that is in communication with a source of air to be tested. The pre-concentrator 106(a) also has an exhaust 122(a) from which a particle-poor stream of air is expelled and an air outlet 120(a) from which a particle-rich stream of air is expelled. The air outlet 120(a) of the pre-concentrator 106(a) is in communication with the air inlet 112 of the collector 102 such that the particle-rich stream of air feeds into the collector inlet. The exhaust 122(a) of the pre-concentrator 106(a) is in communication with the inlet 118(b) of the second concentrator 106(b) so that the particle-poor stream of air can be concentrated into another particle-rich stream of air. The second concentrator 106(b) has an air outlet 120(b) in communication with a mixer 108 to reintroduce or recycle the particle-rich air stream into the system at 118(a) and an exhaust 122(b). As shown by broken lines in FIG. 2, the system 200 can have a series of N concentrators, where N is an integer greater than one. That is, a series of N concentrators 106(a)-(N) can be provided such that the exhaust 122(b) of the second concentrator 106(b) is in communication with the inlet of a third concentrator which concentrates the exhaust for recycling via air inlet 118(a), and so on. The Nth concentrator 106(N) also has an exhaust 122(N) to dispose of particle-poor air from the system 200. It will further be appreciated that the exhaust stream 104(b) from the collector 102 can be reintroduced into the system by causing the collector exhaust to communicate with the inlet of one of the concentrators, for example as shown by broken lines in FIG. 2.

When in operation, the pre-concentrator 106(a) of system 200 creates a vacuum and causes air from the space to be sampled to flow through inlet 118(a). As the system 200 operates, the pre-concentrator 106(a) then creates two streams of air. The first is a stream of particle-rich air that is sent through the pre-concentrator outlet 120(a) and into the collector 102. The pre-concentrator 106(a) sends a second stream of air through its exhaust 122(a). The second stream of air is particle-poor as compared to the concentrated stream sent to the collector 102. The second concentrator 106(b) receives this particle poor exhaust air stream, and concentrates it just as the pre-concentrator 106(a) did to the original air sample. The second concentrator 106(b) then re-circulates the particle-rich air back into the system 200 through outlet 120(b) while ridding the system 200 of particle-poor air through the exhaust 122(b). The outlet 120(b) of the second concentrator 106(b) is in communication with the inlet 118(a) of the pre-concentrator 106(a), thereby allowing the particle-rich air to be re-circulated within the system 200. The outlet 116 of the collector 102 is fed back into the system 200, whereas outlet 116 is in communication with inlet 118(b) of the second concentrator 106(b).

Accordingly, as the system 200 operates, a steady stream of air sample is taken in through the air inlet 118(a) of the pre-concentrator 106(a) while a steady stream of particle-poor air is exhausted through the exhaust 122(b) of the second concentrator 106(b). As the series of concentrators operate in this way, the system 200 assures that the particle content of the air flowing through outlet 120(a) and received by the collector 102 is maximized. Recycling particles through the pre-concentrator 106(a) significantly enhances the ability of the pre-concentrator 106(a) to transport the particles of an aerosol release event to the collector 102.

Again, to quantify the impact of recycling according to the present invention on the ability of the system to transport the aerosol release event to the collector 102, the systems can be described using overall system material (mass) balances. Materials must be conserved within a system and, as such, equations can be formulated that account for the inlets and outlets. These equations are written as steady-state material balances over the system 200 as a whole. Once the mathematical construct is established, various metrics can be analyzed. These include the boost in system 200 performance and the fraction of the aerosol release event that is transported to the detector/collector for the original and recycling systems.

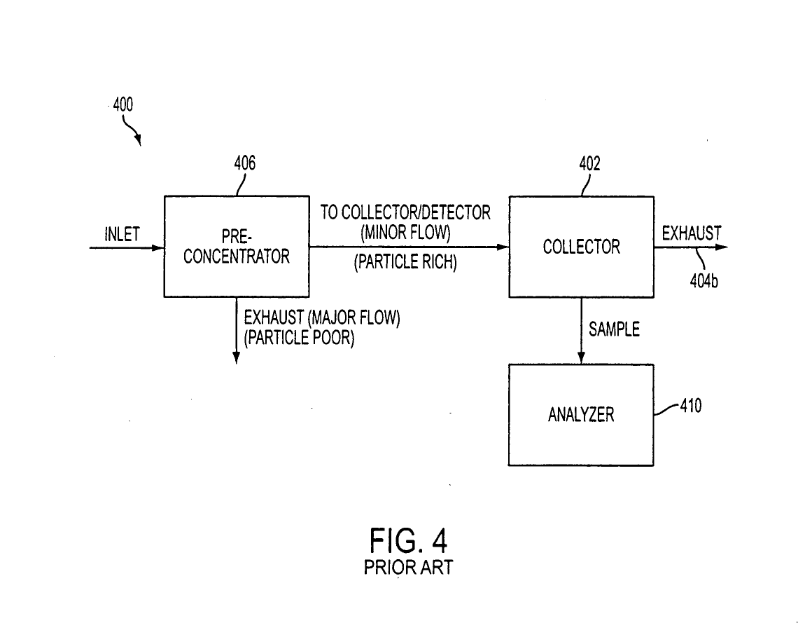

FIG. 4 shows another prior art system 400 in which a pre-concentrator 406 is disposed upstream of a collector inlet 412 to provide a concentrated stream of air to be tested to the collector 402, which provides a sample containing particles for testing by the device 410 and an exhaust stream 404(b). Applying the foregoing mathematical approach to the prior art detection system shown in FIG. 4, and assuming no aerosol loss by deposition in the lines, the fraction of the aerosol release event sent to the collector by the pre-concentrator, f_{Trans}, is simply equal to the separation efficiency of the pre-concentrator, ξ_{PC}, as given in Eqn. 1, below:

f_{Trans}=ξ_{PC} Eqn. 1

Applying the same approach to the biohazard detection system shown in FIG. 2, the fraction of the aerosol release event transported to the collector by the pre-concentrator, f_{Trans}, the function of the separation efficiencies of the pre-concentrator, ξ_{PC}, and second concentrator, ξ_{SC}, as given in Eqn. 2, below:

$${f}_{Trans} = \frac{{ξ}_{PC}}{1 - {ξ}_{SC} ( 1 - {ξ}_{PC} )} \qquad Eqn . 2$$

Note that in the absence of the second concentrator (i.e. no recycling) Eqn. 2 reduces to Eqn. 1.

A simple parameter study, shown in Table 1, reveals some noteworthy findings. First, even for a system with an efficient pre-concentrator (e.g. ξ_{PC}=0.9), recycling can enhance the process such that nearly the entire aerosol release event would be transported to the detector/collector. For systems with moderate to poorly efficient pre- and second concentrators, the addition of a recycling line can boost the fraction transported, f_{Trans}, by 15-25%. Lastly, for systems that have an “inefficient” pre-concentrator (e.g. ξ_{PC}=0.5), an efficient second concentrator can significantly boost (upwards of 40%) the fraction transported.

| | ξ_{PC} | | | | |
|---|---|---|---|---|---|
| | 0.9 | 0.8 | 0.7 | 0.6 | 0.5 |
| ξ_{SC} | f_{Trans} | f_{Trans} | f_{Trans} | f_{Trans} | f_{Trans} |
| 0.9 | 0.989 | 0.976 | 0.959 | 0.938 | 0.909 |
| 0.8 | 0.978 | 0.952 | 0.921 | 0.882 | 0.833 |
| 0.7 | 0.968 | 0.930 | 0.886 | 0.833 | 0.769 |
| 0.6 | 0.957 | 0.909 | 0.854 | 0.790 | 0.714 |
| 0.5 | 0.947 | 0.889 | 0.824 | 0.750 | 0.667 |

Reducing the theory to practice, the flow rates entering the pre- and recycling concentrators would differ only modestly. Consequently, one would most likely employ concentrators of the same design that would have nearly the same efficiency. One possible benefit of using such a recycling system then becomes being able to use two cheaper/less power consuming concentrators in place of one more expensive/consumptive concentrator. For example, employing a recycling system that uses two concentrators that have an efficiency of roughly 0.7 would yield an equivalent fraction transported as that of one using one concentrator with an efficiency of roughly 0.90.

In the case of N concentrators, the additional concentrators act as a dampening system to transport the particles to the collector/detector, much like resistors in series. One can mathematically describe such a system, e.g., as shown in FIG. 2, and arrive at an expression for f_{Trans}, Eqn. 3:

$${f}_{Trans} = \frac{{ξ}_{PC}}{{ξ}_{PC} + {\prod }_{j = 1}^{N + 1} ( 1 - {ξ}_{j} )} , where {ξ}_{1} = {ξ}_{PC} \qquad Eqn . 3$$

where N is the number of additional concentrators, and the pre-concentrator is denoted by j=1.

As with the system with one additional concentrator/recycling line, in practice one could deploy concentrators of the same design. As the flow rates would be similar, the efficiency would be roughly equivalent. Of course, as the number of additional concentrators increases, the flow rates could become significantly different, resulting in differing concentrator efficiencies. For a simple demonstration, this effect is ignored in order to assess the impacts of adding multiple concentrators. If one assumes that all the concentrators have the same efficiency, then Eqn. 3 simplifies to:

$${f}_{Trans} = \frac{ξ}{ξ + {( 1 - ξ )}^{N + 1}} , where {ξ}_{j} = {ξ}_{PC} = ξ \qquad Eqn . 4$$

The boost efficiency, β, would merely be the difference in f_{Trans} for a recycling system versus a system without recycling:

$$β = \frac{ξ}{ξ + {( 1 - ξ )}^{N + 1}} - ξ \qquad Eqn . 5$$

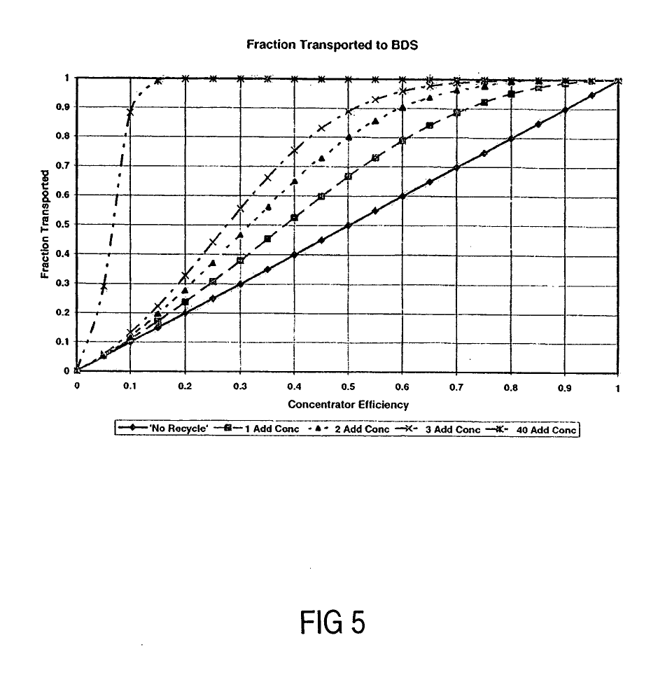

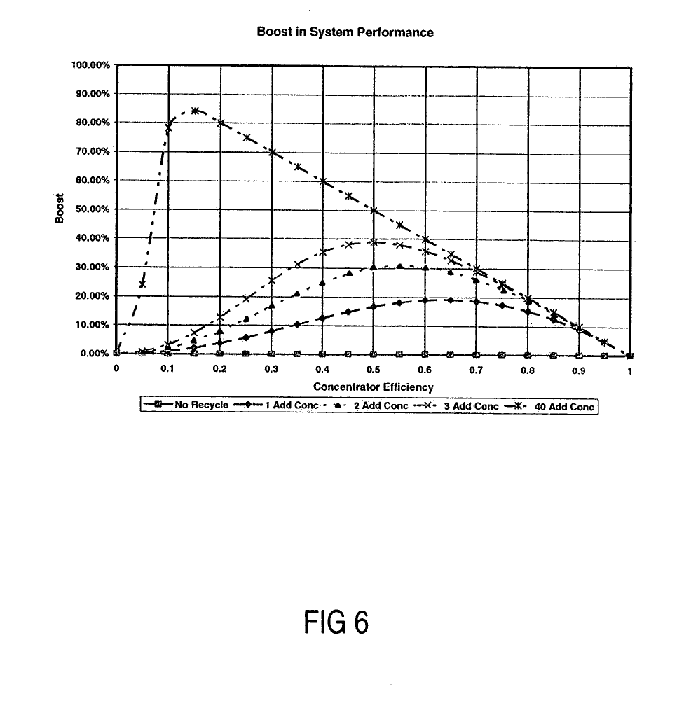

A parameter study can then be conducted to gain insight into how multiple concentrators of varying efficiencies can impact the fraction of the aerosol release event that is sent to the collector/detector. FIGS. 5 and 6 illustrate the parameter study in which three simple conclusions can be drawn. First, as more concentrators are added to the system, the concentrators can be less efficient to maintain the same fraction transported, as shown in FIG. 5. In addition, as the number of concentrators increases, the concentrator efficiency that would yield −100% transport decreases. Lastly, as with all dampening systems, for each system of concentrators in series, there is a concentrator efficiency value in which the system will have a maximum boost in performance. As shown in FIG. 6, the concentrator efficiency that yields the maximum boost in performance decreases with increasing number of concentrators.

This maximum boost in system performance can be numerically computed by finding the local maxima (setting the derivative of Eqn. 5 with respect to ξ equal to zero). Doing so yields the following expression:

$$1 = \frac{{( 1 - ξ )}^{N + 1} + ξ ( N + 1 ) {( 1 - ξ )}^{N}}{{[ ξ + {( 1 - ξ )}^{N + 1} ]}^{2}} \qquad Eqn . 6$$

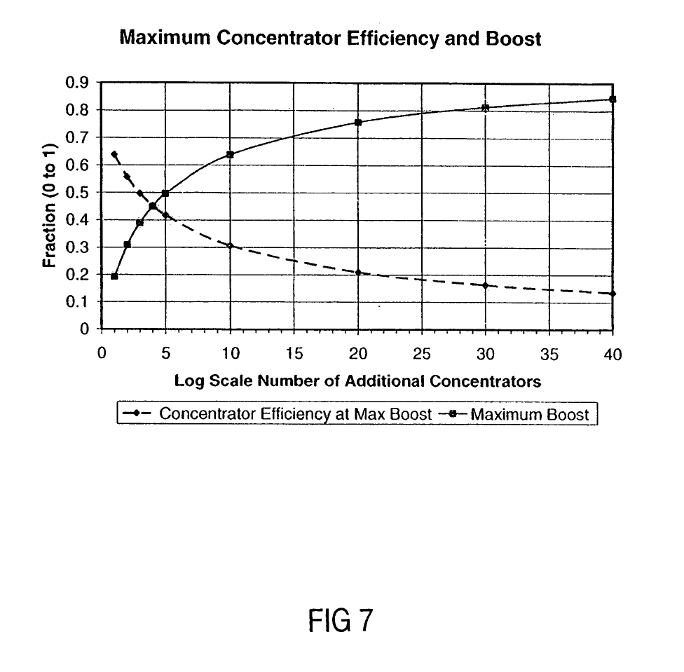

Given the number of additional concentrators, N, the concentrator efficiency that yields the maximum boost in system performance can be computed numerically. Doing so for a growing number of additional concentrators, FIG. 7 shows that the boost in the system performance asymptotically approaches −0.85, while the concentrator efficiency that yields the maximum boost asymptotically approaches −0.15. This means that theoretically, given a large number of additional concentrators, the system could pass all of the particles in an aerosol release event onto the collector/detector.

Of course, these are theoretical limits. A real system would have additional losses due to deposition in the lines that the aerosols flow through. Also for a real system, multiple identical concentrators in series will cause the flow rates through the concentrators to decrease and thereby impact the concentrator efficiencies. Additionally, there are the obvious cost and operational inefficiencies associated with the use of many concentrators in series rather than a single unit. Regardless, the analysis shows that concentrating and recycling particles in the pre-concentrator exhaust stream (by employing one or more additional concentrators) has the potential to significantly increase the fraction of the aerosol release event transported to the collector/detector system. This would provide obvious system performance advantages (most notably increased system sensitivity), and allow for flexibility in design and selection of pre-concentrators.

While various embodiments of the present invention have been described above, it should be understood that they have been presented by way of example only, and not limitation. For example, while use of a wet wall cyclone-type collector is described herein, it will be appreciated that other types of collectors can be used, such as, but not limited to, dry cyclone, dry filter electrostatic and impaction-based collectors. Similarly, while use of a volume-reduction concentrator is described herein, it will be appreciated that various types of aerosol concentrators can be used, including, but not limited to, inertial (cyclone or impaction) and electrostatic concentrators. Furthermore, the detector can be any type of device capable of detecting the presence of a biohazard, including, but not limited to, devices utilizing polymerase chain reaction (PCR) technology, electro-chemoluminescence (ECL), etc. Commercially available collectors and concentrators often have fans or blowers to draw air into the device. It will be appreciated, however, that one or both of the collector and concentrator can be provided without a fan or blower, relying on the other or a separate fan or blower to induce air to flow through the system. It will also be appreciated that the present invention can be used to enhance the performance of existing biohazard detection systems with particle collectors such as the systems described in U.S. Patent Application Publications Nos. 2004/0063197 and 2004/0063198, the contents of which are incorporated herein by reference, by arranging one or more concentrators in relation to the collector as described herein.

Thus, the breadth and scope of the present invention should not be limited by any of the above-described exemplary embodiments, but should be defined only in accordance with the following claims and their equivalents.

1. A system comprising:
a collector including a first fluid inlet in communication with a first stream of fluid having a first concentration of particles to be tested, a particle collection mechanism configured to receive fluid from said first fluid inlet and to remove particles from the fluid for testing, and a first fluid outlet configured to expel at least some of the fluid from said particle collection mechanism as a second stream of fluid having a second concentration of particles lower than the first concentration of particles; and
a concentrator including a second fluid inlet in communication with said first fluid outlet to receive the second stream of fluid, a particle concentration mechanism configured to produce from the second stream of fluid a third stream of fluid with a third concentration of particles and a fourth stream of fluid with a fourth concentration of particles lower than the third concentration of particles, and a second fluid outlet communicating the third stream of fluid from said particle concentration mechanism with said first fluid inlet such that the third stream of fluid is combined with the first stream of fluid entering said collector.

2. The system of claim 1 further comprising a mixer configured to combine the third stream of fluid with the first stream of fluid.

3. The system of claim 2 wherein said mixer includes a venturi manifold.

4. The system of claim 1 further comprising a vacuum source in communication with at least one of said collector and said concentrator o cause fluid to flow through said system.

5. The system of claim 1 wherein at least one of said collector and said concentrator includes a vacuum source configured to cause fluid to flow through said system.

6. The system of claim 1 further comprising a testing instrument configured to receive particles from said collector and to test the particles for the presence of a biohazard.

7. The system of claim 1 wherein said concentrator further includes a third fluid outlet configured to expel the fourth stream of fluid from said concentrator.

8. The system of claim 7 further comprising a second concentrator including a third fluid inlet in communication with said third fluid outlet to receive the fourth stream of fluid, a second particle concentration mechanism configured to produce from the fourth stream of fluid a fifth stream of fluid with a fifth concentration of particles and a sixth stream of fluid with a sixth concentration of particles lower than the fifth concentration of particles, and a fourth fluid outlet communicating the fifth stream of fluid from said second particle concentration mechanism with said first fluid inlet such that the fifth stream of fluid is combined with the first stream of fluid entering said collector.

9. A system comprising:
a first concentrator including a first fluid inlet in communication with a first stream of fluid having a first concentration of particles to be tested, a first particle concentration mechanism configured to produce from the first stream of fluid a second stream of fluid with a second concentration of particles and a third stream of fluid with a third concentration of particles lower than the second concentration of particles, a first fluid outlet configured to expel the second stream of fluid from said particle concentration mechanism, and a second fluid outlet configured to expel the third stream of fluid from said particle concentration mechanism;
a second concentrator including a second fluid inlet in communication with said second fluid outlet to receive the third stream of fluid, a second particle concentration mechanism configured to produce from the third stream of fluid a fourth stream of fluid with a fourth concentration of particles and a fifth stream of fluid with a fifth concentration of particles lower than the fourth concentration of particles, and a third fluid outlet configured to expel the fourth stream of fluid from said second particle concentration mechanism; and
a collector including a third fluid inlet in communication with said first fluid outlet to receive the second stream of fluid from said first concentrator, a particle collection mechanism configured to remove particles from the fluid for testing, and a fourth fluid outlet configured to expel at least some of the fluid from said particle collection mechanism as a sixth stream of fluid having a sixth concentration of particles;
wherein said third fluid outlet is in communication with said first fluid inlet such that the fourth stream of fluid from said second concentrator is combined with the first stream of fluid entering said first concentrator.

10. The system of claim 9 further comprising a mixer configured to combine the fourth stream of fluid with the first stream of fluid.

11. The system of claim 10 wherein said mixer includes a venturi manifold.

12. The system of claim 9 further comprising a vacuum source in communication with at least one of said collector, said first concentrator and said second concentrator to cause fluid to flow through said system.

13. The system of claim 9 wherein at least one of said collector and said concentrator includes a vacuum source configured to cause fluid to flow through said system.

14. The system of claim 9 further comprising a testing instrument configured to receive particles from said collector and to test the particles for the presence of a biohazard.

15. The system of claim 9 wherein said second concentrator further includes a fifth fluid outlet configured to expel the fifth stream of fluid from said concentrator.

16. The system of claim 9 wherein said fourth fluid outlet is in communication with at least one of said first and third fluid inlets.

17. A method comprising the steps of:
receiving in a collector a first stream of fluid having a first concentration of particles;
collecting particles from the first stream of fluid using the collector;
expelling at least some of the fluid from the collector as a second stream of fluid having a second concentration of particles lower than the first concentration of particles;
receiving the second stream of fluid in a concentrator;
using the concentrator to produce from the second stream of fluid a third stream of fluid with a third concentration of particles greater than the second concentration of particles and a fourth stream of fluid with a fourth concentration of particles lower than the third concentration of particles; and
combining the third stream of fluid with the first stream of fluid.

18. The biohazard detection method of claim 17, further comprising the step of testing particles collected during said collecting step for the presence of a biohazard.

19. A method comprising the steps of:
receiving in a first concentrator a first stream of fluid having a first concentration of particles;
using the first concentrator to produce from the first stream of fluid a second stream of fluid with a second concentration of particles greater than the first concentration of particles and a third stream of fluid with a third concentration of particles lower than the second concentration of particles;
receiving in a second concentrator the third stream of fluid;
using the second concentrator to produce from the third stream of fluid a fourth stream of fluid with a fourth concentration of particles greater than the third concentration of particles and a fifth stream of fluid with a fifth concentration of particles lower than the fourth concentration of particles;
combining the fourth stream of fluid with the first stream of fluid received by the first concentrator;
receiving in a collector the second stream of fluid produced by the first concentrator; and
collecting particles from the second stream of fluid using the collector.

20. The biohazard detection method of claim 19, further comprising the step of testing particles collected during said collecting step for the presence of a biohazard.
